(19) United States
(12) Patent Application Publication
(10) Pub. No.: US 2011/0129540 A1
(43) Pub. Date: Jun. 2, 2011

(54) Polymer of acrylic or methacrylic type comprising alpha-tocopherol grafts

(75) Inventors: You-Ping CHAN, Ternay (FR); Olivier BREYNE, Lyon (FR); Alain CONSTANCIS, Lyon (FR)

(73) Assignee: Flamel Technologies, Venissieux (FR)

(21) Appl. No.: 12/955,076

(22) Filed: Nov. 29, 2010

Related U.S. Application Data
(60) Provisional application No. 61/265,092, filed on Nov. 30, 2009.

(30) Foreign Application Priority Data
Nov. 26, 2009 (FR) 09 58413
Nov. 26, 2010 (IB) PCT/IB2010/055440

(51) Int. Cl. A61K 9/14; C08F 8/12; C08F 8/32; A61K 38/00; B82Y 5/00

(52) U.S. Cl. 424/497; 525/132; 424/501; 514/1.1; 977/773

(57) ABSTRACT

The present invention relates to a novel family of polymers of acrylic and/or methacrylic type, comprising alpha-tocopherol grafts, capable of forming nanoparticles in an aqueous medium at a neutral pH.

It also relates to the use of such nanoparticles, non-covalently combined with an active ingredient, in particular an active ingredient of low or average aqueous solubility, for conveying, solubilizing and/or increasing the aqueous solubilization of said active ingredient.

The invention relates to a polymer having a linear backbone of acrylic and/or methacrylic type comprising alpha-tocopherol grafts linked to said backbone. These polymers form nanoparticles in water, which are capable of combining active ingredients of various kinds, and prove more particularly advantageous for purposes of increasing the aqueous solubility of active ingredients.

As described hereafter, a large number of active ingredients, whether therapeutic, prophylactic or cosmetic, can pose problems in terms of formulation with regard to an aqueous solubility which is considered inadequate.

In particular, it proves very difficult to formulate weakly water-soluble active ingredients in a form compatible with administration by oral route, a route particularly valued for the administration of active ingredients, in particular as regards patient comfort and compatibility with a wide variety of formulations.

Thus, the active ingredients, AIs, of class II or class IV of the biopharmaceutical classification mostly have an oral bioavailability which is limited by their low solubility. In particular, paclitaxel, a natural taxoid, widely used for the treatment of tumours is representative of these weakly water-soluble active ingredients. The very low water-solubility of this compound, less than 1 μg/ml, makes its formulation difficult.

In this context, the development of additives making it possible to increase the aqueous solubility of active ingredients is of considerable interest.

Ideally a solubilization additive must have several essential characteristics.

Firstly, it must, for obvious reasons, have a high solubilizing power. Therefore, it is necessary for the polymer to solubilize a large enough quantity of AI. This has two advantages. This ability makes it possible to minimize the quantity of additive, which can be crucial for tolerance in the case of a parenteral form. Moreover, a high solubility makes it possible to make the single dose easy to administer to the patient, whether by oral or parenteral route.

On the other hand, it is advantageous for the formulation of the active ingredient with a solubilization additive to have a low viscosity. Thus, for an active ingredient intended for parenteral administration, the viscosity of the suspension containing the active ingredient and the solubilizing agent must be low enough to allow easy injection through a needle with a small diameter, for example a 27- to 31-gauge needle. In fact, even in the case of oral administration of an AI contained in a tablet, a low viscosity of the suspension solubilizing the active ingredient remains a decisive advantage for the production stages of microparticles, tablets or any other pharmaceutical form known to a person skilled in the art. This low viscosity requirement is particularly restrictive as it limits the acceptable quantity of solubilizing additive and excludes the use of high molecular mass polymer-type additives which are highly water-soluble but have high viscosities.

The development of a solubilization additive allowing the solubilization of active ingredients in a high enough concentration, at the same time meeting all the abovementioned criteria is difficult.

Several alternatives have already been proposed in an attempt to make up for the lack of bioavailability of the weakly water-soluble active ingredients. Among the latter, a particularly useful alternative utilizes micellar solutions. Thus polymeric micelles formed by amphiphilic copolymers, for example PLGA-PEG di-block copolymers, are known. In this formulation method, the active ingredient is solubilized within the hydrophobic PLGA core of the micelles.

However, this approach has in particular two limitations: on the one hand, a moderately soluble AI such as for example a peptide of average solubility, can be difficult to solubilize in the hydrophobic core and on the other hand, the production method for the nanoparticles comprises a stage of solubilizing the PLGA in a hydrophobic solvent, a stage which must be avoided for certain fragile AIs.

In order to overcome these drawbacks, the Applicant has for some ten years been developing polymers based on polyglutamic acid and comprising various hydrophobic grafts. These polymers are used in particular in the field of the controlled release of proteins such as insulin or interferon alpha. The document WO 03/104303 describes more particularly polymers Of polyaminoacid, and in particular polyglutamic, type comprising the alpha-tocopherol grafts linked by an ester function to the carboxylate in the gamma position of the glutamic acid. These polymers form nanoparticles in water and are capable of combining small molecules or proteins. After administration by injection into the sub-cutaneous tissue, these nanoparticles release proteins over a period which can vary from a few days to two weeks. These polymers are biodegraded by enzymes in vivo.

However, this alternative has the drawback of being particularly expensive, given the particularly high production cost of polyglutamic-type polymers.

Moreover, it would be advantageous to be able to have polymers exhibiting increased resistance to hydrolysis by enzymes present in the intestinal tract.

A need therefore remains to have a more economic alternative of amphiphilic polymers, exhibiting improved resistance to degradation by enzymatic hydrolysis, capable of forming nanoparticles which are stable in aqueous medium, and capable of combining non-covalently in the nanoparticle state with active ingredients, in particular active ingredients of low and average aqueous solubility, and becoming dissociated from them in vivo.

The present invention aims precisely to propose a novel family of polymers, and novel compositions, making it possible to meet all of the above-mentioned requirements.

More precisely, according to one of its aspects, the present invention relates to a polymer having a linear backbone of acrylic and/or methacrylic type to which alpha-tocopherol grafts are linked, characterized in that said alpha-tocopherol grafts are linked to said backbone via a spacer partly formed by at least one hydrolyzable function, and in that the distribution of said grafts on said backbone is random.

Advantageously, these polymers are biocompatible.

Preferably, the polymers according to the invention exhibit a molar grafting rate of alpha-tocopherol groups of less than or equal to 30 molar %.

Moreover, the polymers of the invention are capable of spontaneously forming nanoparticles when they are dispersed in an aqueous medium, in particular with a pH ranging from 5 to 8, and in particular water.

According to another of its aspects, the invention relates to a composition, in particular pharmaceutical, cosmetic, dietetic or phytosanitary, comprising at least one polymer as defined previously.

In particular, a composition according to the invention can comprise at least one active ingredient, in particular an active ingredient of low or average aqueous solubility, said active ingredient being present there in a form non-covalently combined with nanoparticles formed by at least one polymer as defined previously.

A composition of the invention can be capable, in particular, of ensuring a regulated release profile of the active ingredient as a function of time.

According to another of its aspects, the invention also relates to the use of nanoparticles of at least one polymer of the invention, non-covalently combined with an active ingredient, with a view to conveying, solubilizing and/or increasing the aqueous solubilization of an active ingredient, in particular an active ingredient of low or average aqueous solubility.

As will be shown below, the polymers according to the invention are particularly useful with regard to their backbone of acrylic and/or methacrylic type.

First of all, these polymer backbones of acrylic and/or methacrylic type are difficult to degrade by enzymes in vivo.

They are also commercially available and relatively inexpensive.

Thus, the polymers of acrylic or methacrylic type already have numerous uses in the field of galenic forms for administration by oral route. The polymers commonly used, known by their tradename Carbopol® or Carbomer® are crosslinked polymers used most often as viscosifiers, controlled-release matrices or mucoadhesive agents. Other families known by the trade names Eudragit®, Kollicoat® or Eastacryl® are for example thickening emulsions or suspensions. These polymers are commonly used as solid matrices, coating or thickening materials. However, this type of polymer does not make it possible to obtain the properties sought by the Applicant.

On the other hand, polymers of acrylic or methacrylic type according to the invention, i.e. bearing alpha-tocopherol grafts on their polymer backbone, via a spacer formed at least partly by one hydrolyzable function, have never been described.

Plasencia et al., J. Mater. Sci. 1999, 641-648, describe films of copolymers obtained from the radical copolymerization of monomers of 2-hydroxyethyl methacrylate type and monomers of tocopherolmethacrylate type. These copolymers are proposed for tendon cicatrization uses. They form hydrogels which are hydrated in the presence of water (films) and are not dispersible in aqueous phase.

Yasuzawa et al., in the document Makromol. Chem. Rapid. Commun. 1985, 6, 727-731, describe a homopolymer of acrylic type obtained by radical polymerization of an acrylic monomer containing a phosphatidyl function and alpha-tocopherol. The polymer thus obtained is also non-dispersible in aqueous phase.

Kim et al. (U.S. Pat. No. 5,869,703) describe non-ionic alpha-tocopherol monomers comprising an acrylate or methacrylate function. The homopolymers originating from these monomers and prepared by radical polymerization in water are capable of organizing themselves in the form of vesicles of 300 to 1200 nm. These non-ionic vesicles are proposed as antioxidants.

It is to the Applicant's credit to have developed new polymers with a linear backbone of acrylic and/or methacrylic type, and having alpha-tocopherol grafts, which form nanoparticulate systems which are stable in an aqueous phase and are hydrolyzable with respect to the specific type of bonding according to the invention between the alpha-tocopherol grafts and the acrylic and/or methacrylic polymer chain.

Thus, as will be shown by the examples presented below, the polymers of the invention make it possible to significantly increase the solubility of active ingredients which are slightly soluble, or even insoluble in water, and more generally of active ingredients of any kind, in particular of peptide or protein type. Furthermore the advantageously low viscosity of these polymers makes it possible, by increasing their concentration, to increase the quantity of active ingredients in solution.

According to another of its aspects, the present invention relates to a method for the preparation of a polymer as defined previously, characterized in that it comprises at least bringing at least one (meth)acrylic(co)polymer into contact, under conditions favourable to their interaction, with at least one alpha-tocopherol derivative functionalized with a spacer provided at its free end with a function capable of interacting with an acid function of said polymer, said spacer being such that, on completion of the reaction with said (co)polymer, it comprises at least one hydrolyzable function.

By polymer “of acrylic and/or methacrylic type”, is meant polymers the linear backbone or also the main chain of which is formed by acrylic and/or methacrylic acid unit(s) and by methyl, ethyl, propyl or butyl acrylate and/or methacrylate unit(s).

It is understood that the derivatization of the acrylic or methacrylic acid units forming said backbone with at least one alpha-tocopherol unit leads to the conversion of these units to acrylate or methacrylate units.

Of course, the polymer chain can moreover comprise acrylate and/or methacrylate units distinct from those derivatized by a tocopherol group.

For example, acrylate and/or methacrylate units can be derivatized by a polyalkylene glycol unit, as described below, or also by a spacer according to the invention but not functionalized at its free end by a tocopherol unit.

This last case in point can in particular arise when the polymer of the invention is prepared according to a method involving a preliminary stage of grafting the spacer onto the polymer chain, then consecutive grafting of the alpha-tocopherol, the latter not then reacting with all of the grafted spacers.

The main chain of the (co)polymers of acrylic and/or methacrylic type of the invention can comprise acrylic acids and acrylate units or methacrylic acid and methacrylate units, or also a mixture of the two previous alternatives in the case where the main chain is a copolymer formed by copolymerization of at least two distinct monomers, for example of acrylic and methacrylic type.

Preferably, the polymers according to the invention are constituted by acrylic acid and acrylate type units, the latter comprising alpha-tocopherol grafts, as specified above.

The distribution of the units of acrylate and/or methacrylate type bearing alpha-tocopherol grafts is, according to the present invention, such that the polymers thus constituted are polymers of random type.

By “polymer of random type”, is meant that the monomer units of acrylate and/or methacrylate type, bearing alpha-tocopherol grafts, are irregularly distributed within the poly(meth)acrylic chain, independently of the nature of the adjacent units.

According to a particularly preferred embodiment, the molar grafting rate with alpha-tocopherol grafts of the polymer according to the invention is less than or equal to 30 molar %, in particular less than or equal to 20 molar %, in particular greater than or equal to 3 molar %, and preferably comprised between 5 and 10 molar %. In other words, no more than 30% of the acrylic and/or methacrylic units forming the backbone of the (co)polymer according to the invention bear side segments of alpha-tocopherol type.

The alpha-tocopherol can be presented in its D-alpha-tocopherol form (its natural form) or its D,L-alpha-tocopherol form (racemic and synthetic form).

The alpha-tocopherol according to the invention can be of natural or synthetic origin, Preferably, the alpha-tocopherol is of synthetic origin.

As mentioned previously, the linking of the alpha-tocopherol to the main chain of the polymers according to the invention is established via a spacer.

A “spacer” according to the invention represents a chemical entity partly formed by at least one hydrolyzable function. It is therefore bifunctional and different from a simple chemical bond. Similarly, this spacer is different from the reactive unit obtained by direct interaction of an acid function of the polymer backbone with a function carried on the tocopherol backbone.

This hydrolyzable function can be located at the end of said spacer linked to the polymer backbone, at the end of said spacer linked to the alpha-tocopherol group, or within said spacer.

According to a preferred variant, this hydrolyzable function originates from the reaction of a function present on the polymer backbone or a function present on the alpha-tocopherol molecule with a reactive function present on the precursor molecule of the spacer.

This spacer contains at least one carbon atom.

The spacer according to the invention, linking an alpha-tocopherol unit to a (meth)acrylate type unit of the (co)polymer advantageously comprises two hydrolyzable functions, one establishing a covalent bond with the polymer backbone and the other with the alpha-tocopherol unit.

The hydrolyzable function(s) present on the spacer is (are) more particularly an ester, amide, carbonate or carbamate function(s).

According to a preferred embodiment of the invention, the spacer according to the invention is an amino acid residue. Preferably, it is a natural amino acid residue, in particular chosen from alanine, glycine, phenylalanine or leucine.

According to a particularly preferred embodiment, the spacer is an alanine residue.

According to a particularly advantageous embodiment, the polymer according to the invention is a polymer of the following formula (I),

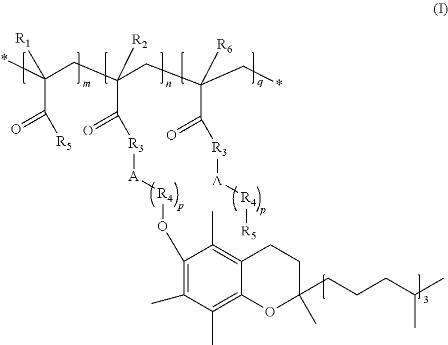

In which:
R₁, R₂ and R₆ represent independently an H or a methyl; —R₃-A-(R₄)ₚ— constitutes the spacer according to the invention;
R₃ represents —NH— or —O—;
A represents a linear C₁ to C₂ alkyl, or a linear or branched C₂ to C₆ alkyl or a methylene substituted by a benzyl group;
p is equal to 0 or 1, and preferably p is equal to 1;
R₄ represents C═O, O—C═O, or NH—C═O;
R₅ represents —OH or —OM, with M representing a cation, or R₅ represents a polyalkylene glycol substituent linked to the polymer via an ester function or an amide function;
m and n are positive integers;
q is equal to 0 or a positive integer, and preferably q is equal to 0;
(m+n+q) varies from 20 to 300,000;
the molar grafting rate of the alpha-tocopherol groups, n/(m+n+q) is less than or equal to 30 molar %,
the order of succession of the two, or even three types of units forming the backbone of the formula (I) being completely random.

According to a particularly advantageous embodiment, the polymer according to the invention is a polymer of the following formula (I′),

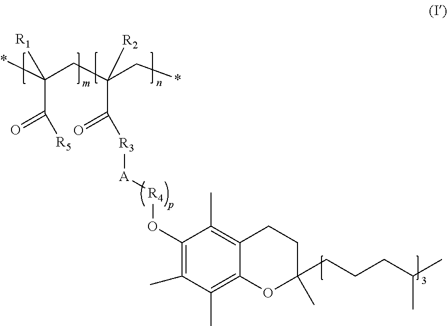

in which R₁ to R₅, m, n and p are as defined previously.

As is shown by the previous definition, the general formulae (I) and (I′) described above should not be interpreted as representing sequenced (or block) copolymers, involving a specific order between the two or three types of represented units forming the backbone. Within the meaning of the invention, the order of succession of the two, or even three types of units, is completely random.

Advantageously, the poly(meth)acrylic polymer contains carboxylic functions which are either neutral (COOH form), or ionized depending on the pH and the composition.

In aqueous solution, the counter-cation can be an inorganic cation such as sodium, calcium, magnesium or ammonium, or an organic cation such as the protonated form of triethylamine, triethanolamine, tri(hydroxymethyl)-aminomethane or a tetraalkylammonium (alkyl being methyl, ethyl, propyl or butyl), or also the protonated form of an amino acid, in particular lysine or arginine.

In particular, in the previous formula (I) or (I′), when p=0, the spacer according to the invention, linking an alpha-tocopherol unit to a (meth)acrylate type unit of the (co)polymer, comprises a single hydrolyzable function linked to the (meth)acrylate unit, which can be an amide function or an ester function.

According to a preferred embodiment, p is equal to 1, the spacer then possessing two hydrolyzable functions.

The polymers of general formula (I) or (I′), preferably of formula (F), in which p is equal to 1, and the hydrolyzable chemical entity —R₃-A-R₄— constitutes an amino acid residue, preferably a natural amino acid residue, are quite particularly suitable for the invention.

According to a particularly preferred embodiment, the polymer according to the invention is a polymer of formula (I) or (I′), preferably of formula (I′), in which —R₃-A-R₄— constitutes an alanine residue.

According to a particular embodiment, the polymer according to the invention has an average molar mass ranging from 2,000 to 1,000,000, and preferably from 5,000 to 50,000.

Advantageously, the polymer is biocompatible.

As specified previously, a (co)polymer according to the invention can moreover bear one or more polyalkylene glycol type grafts, in particular linked to a (meth)acrylate type unit constituting it.

Preferably, the polyalkylene glycol graft is a polyethylene glycol having an average molar mass ranging from 1,000 to 5,000 kDa, which can be represented diagrammatically according to one of the following structures:

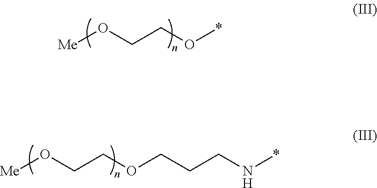

Such grafts are linked to the polymer via an ester (formula II) or amide (formula III) function.

Preferably, the polyalkylene glycol type grafts are used with a molar percentage of grafting varying from 1 to 10%.

As specified above, the polymers considered according to the invention are capable of spontaneously forming nanoparticles when they are dispersed in an aqueous medium with a pH ranging from 5 to 7, in particular water.

Generally, the formation of nanoparticles is due to a self-association of a multitude of polymer chains with segregation of the hydrophobic groups in nanodomains. A nanoparticle can contain one or more hydrophobic nanodomains.

The size of the nanoparticles can vary from 1 to 1,000 nm, in particular from 5 to 500 nm, in particular from 10 to 300 nm, and more particularly from 10 to 200 nm, or even from 10 to 100 nm.

The size of the nanoparticles can be measured by light diffraction.

Method of Preparation

As specified previously, the polymers according to the invention can be obtained according to a method comprising at least bringing, under conditions favourable to their interaction, at least one (meth)acrylic (co)polymer into contact with at least one alpha-tocopherol derivative functionalized with a spacer provided at its free end with a function capable of interacting with an acid function of said polymer, said spacer being such that, on completion of the reaction with said (co)polymer, it comprises at least one hydrolyzable function.

As an example of alpha-tocopherol derivative which functionalized with a spacer according to the invention, mention can be made of alpha-tocopherol leucine, as described for example in the document WO 03/104303.

Alpha-tocopherol glycine and alpha-tocopherol gamma-amino butyrate can also be cited, as described in the document Takata et al., J. Pharm. Sci., 1995, 84, 96-100.

According to a preferred variant, the interaction, or also grafting, of said (co)polymer with said alpha-tocopherol derivative leads to the formation of a hydrolyzable function at the junction between the two entities.

Such a method makes it possible in particular to easily control the grafting rate, and to obtain a polymer of random type.

The grafting of an alpha-tocopherol derivative functionalized according to the invention, with an acid function of the (meth)acrylic (co)polymer is within the competence of a person skilled in the art.

The two types of compounds are brought into contact in a weight or molar ratio adjusted so that the grafting is carried out advantageously at a rate of less than 30 molar %.

For example, the establishment of a covalent bond between the two entities can easily be achieved by reaction of the poly(meth)acrylic (co)polymer with the alpha-tocopherol derivative functionalized by the spacer, in the presence of a carbodiimide as coupling agent and, preferably, a catalyst such as 4-dimethylaminopyridine, and in an appropriate solvent such as dimethylformamide (DMF). The carbodiimide is for example, diisopropylcarbodiimide. The grafting rate is chemically controlled by the stoichiometry of the constituents and reagents and/or the reaction time.

According to a particularly preferred embodiment, the alpha-tocopherol derivative is functionalized by a spacer having at its free end a primary amine function, forming an amide bond after grafting to said (meth)acrylic type(co)polymer.

According to a preferred variant, said spacer is an amino acid residue. The grafting of the corresponding alpha-tocopherol derivative to the (meth)acrylic (co)polymer is then carried out via the reaction of the free amine function of said spacer with the acid function of the (meth)acrylate unit of said (co)polymer.

As regards the functionalization of the alpha-tocopherol by the spacer according to the invention, this is also within the competence of a person skilled in the art.

For example, it can be carried out by reaction, in the presence of a coupling agent and a catalyst, of the alpha-tocopherol with a bifunctional reagent, a precursor of the spacer considered according to the invention, and a single one of whose functions is reactive with respect to the alpha-tocopherol. The second function not dedicated to the reaction with the alpha-tocopherol is then generally used in a form protected with a protective group. On completion of the coupling reaction, this second function, if protected by a protective group, is deprotected in order to allow its interaction with an acid function of the (meth)acrylic (co)polymer.

For example, the functionalization of the alpha-tocopherol can be carried out by reaction of the alpha-tocopherol with a reagent comprising, on the one hand, a protected amine or alcohol function, and on the other hand, a carboxylic function, capable of reacting with the alpha-tocopherol in the presence of a coupling agent and a catalyst. Once deprotected, the amine or alcohol function then allows the grafting of the spacer functionalized by the alpha-tocopherol onto the polymer. By way of example, when the spacer is alanine, the Boc-Alanine derivative is used as reagent in order to prepare the tocopheryl alanine derivative.

According to another variant, it is also possible to graft the “spacer” onto the polymer first and then graft the alpha-tocopherol with the same chemistry described previously and well known to a person skilled in the art. In this case, it is possible to have, in the polymer of the invention, some of the spacers in a form not functionalized by the alpha-tocopherol.

As specified previously, the nanoparticles formed by at least one polymer as described previously can easily combine non-covalently with active ingredients.

Active Ingredients

The present invention advantageously makes it possible to increase the aqueous solubilization of active ingredients in general, and of active ingredients of average or low aqueous solubility in particular.

The invention thus proves quite particularly advantageous with respect to weakly water-soluble active ingredients.

Within the meaning of the present invention, an active ingredient of low water-solubility is a compound possessing a solubility of less than 1 g/l, in particular less than 0.1 g/l, in pure water, measured at ambient temperature, i.e. approximately 25° C.

Within the meaning of the invention, a pure water is a water with a pH close to neutrality (between pH 5 and pH 8) and devoid of any other solubilizing compound known to a person skilled in the art, such as surfactants or polymers (PVP, PEG).

The active ingredients considered according to the invention are advantageously biologically active compounds which can be administered to an animal or human organism.

According to an embodiment variant, these active ingredients are non-peptide.

Generally, an active ingredient according to the invention can be any molecule of therapeutic, cosmetic, prophylactic or imaging interest.

Thus, in the pharmaceutical field, the active ingredients with low water-solubility according to the invention can in particular be chosen from the anti-cancer agents, beta-blockers, anti-fungal agents, steroids, anti-inflammatory agents, sex hormones, immunosuppressors, anti-viral agents, anaesthesics, anti-emetics and antihistamines.

More particularly, there may be mentioned as representing specific active ingredients with low water-solubility, the taxane derivatives such as paclitaxel, nifedipine, carvedilol, camptothecin, doxorubicin, cisplatin, 5-fluorouracil, cyclosporine A, PSC 833, amphotericin B, itraconazole, ketoconazole, betamethasone, indomethacin, testosterone, estradiol, dexamethasone, prednisolone, triamcinolone acetonide, nystatin, diazepam, amiodarone, verapamil, simvastatin, rapamycin and etoposide.

According to a particular embodiment, the active ingredient considered according to the invention is an active ingredient of therapeutic interest.

According to another particular embodiment, the active ingredient can be chosen from paclitaxel, carvedilol base, simvastatin, nifedipine and ketoconazole.

According to an embodiment of the invention, the active ingredient can be a molecule of average aqueous solubility the solubility of which can be increased by a composition according to the invention.

By molecule of average aqueous solubility is meant a molecule the solubility of which in pure water, measured as indicated previously, at ambient temperature is comprised between 1 and 30 g/L, in particular comprised between 2 and 20 g/L of pure water.

According to a particular embodiment, the active ingredients considered according to the invention are of peptide or protein type.

With respect to peptides or proteins, their solubilization, although essential can be less restrictive as the doses can be lower. As illustrative and non-limitative examples of active ingredients according to the invention, there may be in particular mentioned:
the proteins or glycoproteins, in particular interleukins, erythropoietin or cytokines,
the proteins linked to one or more polyalkyleneglycol chains [preferably polyethyleneglycol (PEG): “PEGylated proteins”],
the peptides,
the polysaccharides,
the liposaccharides,
the oligonucleotides, the polynucleotides,

and mixtures thereof.

More particularly, there may be mentioned the following peptides or proteins: insulin or analogues, GLP-1 derivatives, exenatide, cyclosporine, interferons, interleukins and growth hormone.

Combination of the Polymer with an Active Ingredient

The active ingredients can combine spontaneously with the polymer as described previously.

The terms “combination” or “combined” used to qualify the relationships between one or more active ingredients and a polymer according to the invention, signify that the active ingredient or ingredients are combined with the polymer(s) by non-covalent physical interactions, in particular hydrophobic interactions, and/or electrostatic interactions and/or hydrogen bonds and/or via a steric encapsulation by the polymers of the invention.

This combination is generally a matter of hydrophobic and/or electrostatic interactions, achieved by the polymer units, in particular hydrophobic or ionized, capable of generating this type of interaction.

The techniques of combining one or more active ingredients with the polymers according to the invention are similar to those described in particular in the U.S. Pat. No. 6,630,171.

No stage of chemical crosslinking of the particles obtained is provided. The absence of chemical crosslinking makes it possible to avoid the chemical degradation of the active ingredient during the stage of crosslinking the particles containing the active ingredient. Such a chemical crosslinking is in fact generally carried out by activation of polymerizable entities and involves potentially denaturing agents such as UV radiation or glutaraldehyde.

The combination according to the invention of the active ingredient and the polymer can in particular be carried out according to the following embodiments.

In a first embodiment, the active ingredient is dissolved in an aqueous solution and mixed with an aqueous suspension of the polymer.

In a second embodiment, the active ingredient in the form of powder is dispersed in an aqueous suspension of the polymer and the mixture is stirred until a homogeneous limpid suspension is obtained.

In a third embodiment, the polymer is introduced in the form of powder into a dispersion or an aqueous solution of the active ingredient.

In a fourth embodiment, the active ingredient and/or the polymer is dissolved in a solution containing an organic solvent which is miscible with water such as ethanol or isopropanol. The procedure according to embodiments 1 to 3 above is then followed. Optionally, this solvent can be eliminated by dialysis or any other technique known to a person skilled in the art.

For all of these embodiments, it can be advantageous to facilitate the interaction between the active ingredient and the polymer using ultrasound or a rise in temperature.

Microparticles

According to a particular embodiment, the nanoparticles combined non-covalently with said active ingredient can be utilized in a composition according to the invention, in the form of microparticles.

According to a first embodiment, these microparticles can be obtained by agglomeration of nanoparticles of the invention according to the methods known to a person skilled in the art, for example, as a non-limitative illustration, by flocculation, atomization, freeze-drying or coacervation.

The nanoparticulate or microparticulate forms, in the neutral or ionized form, can, more generally, be used alone or in a liquid, solid or gel composition and in an aqueous or organic medium.

The microparticulate forms generally have a core containing said nanoparticles, and at least one coating.

Advantageously, the polymers of the invention can also be used as coating materials alone or in combination with other polymers in particular as described hereafter.

According to another embodiment, the microparticles have a core containing said nanoparticles and at least one coating layer influencing a regulated release profile of said active ingredient as a function of the pH, said coating layer being formed by a material comprising at least one polymer A which is insoluble in water with a pH less than and soluble in water with a pH greater than 7, combined with at least one hydrophobic compound B.

The controlled release of the nanoparticles, as a function of the pH, from the microparticles is ensured by the coating surrounding the core of each reservoir particle. This coating is designed in order to release the active ingredient and the (meth)acrylic (co)polymer at very specific sites of the gastro-intestinal tract corresponding for example to the absorption windows of the active ingredient in the gastro-intestinal tract.

Due to the nature of this coating, the microparticles considered according to the present invention can thus advantageously have a double release mechanism as a function of the time and the pH.

By this expression is meant that they possess the following two specificities. Below the solubilizing pH value of the polymer A forming the coating of these microparticles, they release only a very limited quantity of nanoparticles. On the other hand, when they are present in the intestine or a comparable medium, they ensure an effective release of the nanoparticles. This release can then be carried out advantageously in less than 24 hours, in particular in less than 12 hours, in particular in less than 6 hours, in particular in less than 2 hours or even in less than 1 hour.

In the case of active ingredients having a very narrow absorption window, for example limited to the duodenum or Peyer's patches, the release time of the nanoparticles is less than 2 hours and preferably less than 1 hour.

Thus, a composition according to the invention promotes the release, in a first phase, of the active ingredient combined with the nanoparticles of polymer(s) of the invention then the dissociation, in a second phase, of the active ingredient from said nanoparticles.

The size of the microparticles considered according to this variant of the invention is advantageously less than 2,000 μm, in particular varies from 100 to 1,000 μm, in particular from 100 to 800 μm and in particular from 100 to 500 μm.

The size of the microparticles can be measured by laser granulometry.

According to this embodiment variant, the coating of the nanoparticles can be formed by a composite material obtained by mixing:
at least one compound A which is insoluble in water at a pH of less than 5 and soluble in water at a pH greater than 7;
at least one hydrophobic compound B;
and optionally at least one plasticizer, one antioxidant and/or other conventional excipients.

Polymer A

By way of non-limitative illustration of polymers A which are suitable for the invention, i.e. insoluble in water at a pH of less than 5 and soluble in water at a pH greater than 7, there can in particular be mentioned:
methacrylic acid and methyl methacrylate copolymer(s),
methacrylic acid and ethyl acrylate copolymer(s),
cellulose acetate phthalate (CAP),
cellulose acetate succinate (CAS),
cellulose acetate trimellitate (CAT),
hydroxypropylmethylcellulose phthalate (or hypromellose phthalate) (HPMCP),
hydroxypropylmethylcellulose acetate succinate (or hypromellose acetate succinate) (HPMCAS),
carboxymethylethylcellulose,
shellac gum,
polyvinyl acetate phthalate (PVAP),
and mixtures thereof.

According to a preferred embodiment of the invention, this polymer A is chosen from methacrylic acid and methyl methacrylate copolymer(s), methacrylic acid and ethyl acrylate copolymer(s) and mixtures thereof.

The polymers A dissolve in water at a given pH value, comprised between 5 and 7, this value varying as a function of their intrinsic physico-chemical characteristics, such as their chemical nature and their chain length.

For example, the polymer A can be a polymer the solubilizing pH value of which is:
5.0, such as for example hydroxypropylmethylcellulose phthalate and in particular that marketed under the name HP-50 by Shin-Etsu,
5.5, such as for example hydroxypropylmethylcellulose phthalate and in particular that marketed under the name HP-55 by Shin-Etsu or methacrylic acid and ethyl acrylate copolymer 1:1 and in particular that marketed under the name Eudragit L100-55 by Evonik,
6.0 such as for example a methacrylic acid and methyl methacrylate copolymer 1:1 and in particular that marketed under the name Eudragit L100 by Evonik,
7.0 such as for example a methacrylic acid and methyl methacrylate copolymer 1:2 and in particular that marketed under the name Eudragit S100 by Evonik.

All of these polymers are soluble at a pH value above their solubilizing pH.

The coating is advantageously composed of 25 to 90%, in particular 30 to 80%, in particular 35 to 70%, or even 40 to 60% by weight of polymer(s) A relative to its total weight.

More preferably, the polymer A is a methacrylic acid and ethyl acrylate copolymer 1:1.

Hydrophobic Compound B

According to a first variant, compound B can be selected from the products crystallized in the solid state and having a melting temperature T_{fb}≧40° C., preferably T_{fb}≧50° C., and still more preferably 40° C.≦T_{fb}≦90° C.

More preferably, this compound is then chosen from the following group of products:
vegetable waxes in particular alone or in a mixture with each other, such as those marketed under the trademarks DYNASAN P60; DYNASAN 116;
hydrogenated vegetable oils alone or in a mixture with each other; preferably chosen from the group comprising: hydrogenated cotton seed oil, hydrogenated soya oil, hydrogenated palm oil and mixtures thereof;
mono and/or di and/or tri esters of glycerol and of at least one fatty acid, preferably behenic acid, in particular alone or in a mixture with each other;
and mixtures thereof.

According to this embodiment, the B/A weight ratio can vary between 0.2 and 1.5 and preferably between 0.45 and 1.

More preferably, compound B is hydrogenated cotton seed oil.

Such a coating is in particular described in the document WO 03/30878.

According to a second variant, the compound B can be a polymer which is insoluble in water.

The water-insoluble polymer B is more particularly selected from ethylcellulose, for example marketed under the name Ethocel®, cellulose acetate butyrate, cellulose acetate, ammonia (meth)acrylate copolymers (ethyl acrylate, methyl methacrylate and trimethylammonio ethyl methacrylate copolymer) in particular those marketed under the names Eudragit® RL and Eudragit® RS, poly(meth)acrylic acid esters, in particular those marketed under the name Eudragit® NE and mixtures thereof.

Ethylcellulose, cellulose acetate butyrate and the ammonio (meth)acrylate copolymers in particular those marketed under the names Eudragit RS® and Eudragit RL® are quite particularly suitable for the invention.

The coating of the microparticles can contain 10% to 75%, preferably 15% to 60%, more preferably 20% to 55%, or even 25 to 55% by weight, and still more particularly 30 to 50% polymer(s) B relative to its total weight.

Advantageously, the coating can then be formed, according to this embodiment, from a mixture of the two categories of polymers A and B in a polymer(s) B/polymer(s) A weight ratio greater than 0.25, in particular greater than or equal to 0.3, in particular greater than or equal to 0.4, in particular greater than or equal to 0.5, or even greater than or equal to 0.75.

According to another embodiment variant, the polymer(s) A/polymer(s) B ratio is moreover less than 8, in particular less than 4, or even less than 2 and more particularly less than 1.5.

According to a particular embodiment, the coating of the microparticles is formed by at least one mixture comprising, as polymer A, at least ethylcellulose or cellulose acetate butyrate or the ammonia (meth)acrylate copolymer or a mixture thereof, with, as polymer B, at least one methacrylic acid and ethyl acrylate copolymer or a methacrylic acid and methyl methacrylate copolymer or a mixture thereof.

Apart from the abovementioned two types of compounds A and B, the coating of the nanoparticles according to the invention can comprise at least one plasticizer.

Plasticizer

This plasticizer can in particular be chosen from:
glycerol and its esters, and preferably from the acetylated glycerides, glyceryl-mono-stearate, glyceryl-triacetate, glyceryl-tributyrate,
the phthalates, and preferably from dibutyl phthalate, diethyl phthalate, dimethyl phthalate, dioctyl phthalate,
the citrates, and preferably from acetyl tributyl citrate, acetyl triethyl citrate, tributyl citrate, triethyl citrate,
the sebacates, and preferably from diethyl sebacate, dibutyl sebacate,
the adipates,
the azelates,
the benzoates,
chlorobutanol,
the polyethylene glycols,
the vegetable oils,
the fumarates, preferably diethyl fumarate,
the maleates, preferably diethyl maleate,
the oxalates, preferably diethyl oxalate,
the succinates, preferably dibutyl succinate,
the butyrates,
the cetyl alcohol esters,
the malonates, preferably diethyl malonate,
castor oil,
and mixtures thereof.

In particular, the coating can comprise less than 30% by weight, preferably 1% to 25% by weight, and, still more preferably, 5% to 20% by weight of plasticizer(s) relative to its total weight.

Of course, the coating can comprise various other additional adjuvants used in a standard fashion in the field of coating. These can be, for example:
pigments and colouring agents, such as titanium dioxide, calcium sulphate, precipitated calcium carbonate, iron oxides, natural food colouring agents such as caramels, carotenoids, carmine, chlorophyllins, Rocou (or mulatto), xanthophylls, anthocyans, betanin, aluminium and synthetic food colouring agents such as the yellows No. 5 and No. 6, the reds No. 3 and No. 40, the green No. 3 and Emerald green, the blues No. 1 and No. 2;
fillers, such as talc, magnesium stearate, magnesium silicate;
anti-foaming agents, such as simethicone, dimethicone;
surfactants, such as phospholipids, polysorbates, polyoxyethylene stearates, fatty acid esters and polyoxyethylenated sorbitol, polyoxyethylenated hydrogenated castor oils, polyoxyethylenated alkyl ethers, glycerol
monooleate,
and mixtures thereof.

The coating can be single or multi-layer. According to an embodiment variant, it is made up of a single layer formed by the composite material defined previously.

The formation of the microparticles according to this variant of the invention can be carried out by any conventional technique suitable for the formation of a reservoir capsule the core of which is formed wholly or partly by at least one active ingredient non-covalently combined with nanoparticles of polymer, in particular as defined above.

Preferably, the microparticles are formed by spraying the compounds A and B and if present the other ingredients including the plasticizer(s) generally in the solute state. This solvent medium generally contains organic solvents mixed or not mixed with water. The coating thus formed proves homogeneous in terms of composition as opposed to a coating formed by a dispersion of these same polymers, in a mostly aqueous liquid.

According to a preferred embodiment variant, the sprayed solution contains less than 40% by weight of water, in particular less than 30% by weight of water and more particularly less than 25% by weight of water.

According to another embodiment variant, the nanoparticles non-covalently combined with the active ingredient, can be utilized in a supported form, of microparticle type, in particular on a neutral substrate using one or more binding agents and with one or more conventional excipients.

These microparticles, present in a supported form can be subsequently coated with one or more coating layers, as described previously.

In the case where it is desirable to apply the AI/polymer mixture to a neutral substrate of neutral sphere type, the following procedure can be followed:

A conventional binding agent intended to ensure the cohesion of the layer deposited on the neutral core is added to the homogeneous mixture of active ingredient and polymer.

Such binding agents are in particular proposed in Khankari R. K. et al., Binders and Solvents in Handbook of Pharmaceutical Granulation Technology, Dilip M. Parikh ed., Marcel Dekker Inc., New York, 1997.

The following are quite particularly suitable for the invention as binding agents: hydroxypropylcellulose (HPC), polyvinylpyrrolidone (PVP), methylcellulose (MC) and hydroxypropylmethylcellulose (HPMC).

The deposition of the corresponding mixture is then carried out by the standard techniques known to a person skilled in the art. This may in particular involve spraying the colloidal suspension of the nanoparticles loaded with active ingredients, and containing the binding agent and optionally other compounds, onto the support in a fluidized bed.

Without this being limitative, a composition according to the invention can for example contain, apart from the nanoparticles combined with the active ingredient and the conventional excipients, sucrose and/or dextrose and/or lactose, or also a microparticle of an inert substrate such as cellulose serving as a support for said nanoparticles.

Thus, in a first preferred embodiment of this variant, a composition according to the invention can comprise granulates containing a polymer of the invention, the active ingredient, one or more binding agents ensuring the cohesion of the granulate and various excipients known to a person skilled in the art.

A coating can then be deposited on this granulate by any technique known to a person skilled in the art, and advantageously by spray coating, leading to the formation of microparticles, as described previously.

The composition by weight of a microparticle according to this embodiment is the following:
the content by weight of nanoparticles loaded with active ingredient in the core is comprised between 0.1 and 80%, preferably between 2 and 70% preferably also between 10 and 60%;
the content by weight of binding agent in the core is comprised between 0.5 and 40%, preferably between 2 and 25%;
the content by weight of the coating in the microparticle is comprised between 5 and 50%, preferably between 15 and 35%.

In a second preferred embodiment of this variant, a composition according to the invention can comprise neutral cores around which a layer is deposited containing the active ingredient, the polymer nanoparticles, a binding agent ensuring the cohesion of this layer and optionally different excipients known to a person skilled in the art, for example sucrose, trehalose and mannitol. The neutral core can be a particle of cellulose or sugar or any inert organic or saline compound which lends itself to coating.

The neutral cores thus covered can then be coated with at least one coating layer, to form microparticles, as described previously.

The composition by weight of a particle according to this embodiment is then the following:
the content by weight of nanoparticles loaded with active ingredient in the core is comprised between 0.1 and 80%, preferably between 2 and 70% preferably also between 10 and 60%;
the content by weight of neutral core in the core of the microparticles is comprised between 5 and 50%, preferably between 10 and 30%;
the content by weight of binding agent in the core of the microparticles is comprised between 0.5 and 40%, preferably between 2 and 25%;
the content by weight of the coating in the microparticle is comprised between 5 and 50%, preferably between 15 and 35%.

The present invention also relates to novel pharmaceutical, phytosanitary, food, cosmetic or dietetic preparations based on the compositions according to the invention.

The composition according to the invention can thus be presented in the form of a powder, a solution, a suspension, a tablet or a gelatin capsule.

The composition according to the invention can in particular be intended for the preparation of medicaments.

It can be intended for administration by oral route or by parenteral route.

The invention is better explained by the examples hereafter, given only by way of illustration.

### EXAMPLES

### Example 1

### Synthesis of Polymer 1: Polyacrylic Acid Substituted with Approximately 5 Molar % of Alpha-Tocopherol Grafts Linked Via Alanine

Stage 1: Purification of Commercial Polyacrylic Acid (Degacryl 4779L)

75 g of DEGACRYL 4779L solution (sold by Evonik) are diluted with 1425 g of milli-Q water then diafiltered against 8 volumes of water. The solution obtained is then freeze-dried. The average molar mass Mn, measured by steric exclusion chromatography, is 33.6 kDa in PMMA (polymethyl methacrylate) equivalent and the polydispersity index is 2.4.

Stage 2: Synthesis of Alanine and α-Tocopherol Ester (AlaVE)

22.08 mL of N,N′-Diisopropylcarbodiimide (DIPC) are added dropwise to a solution of 21.1 g of N-Boc alanine, 40 g of α-tocopherol and 0.567 g of dimethylaminopyridine (DMAP) in 400 mL of dichloromethane. After stirring at 20° C. for 22 hours, the reaction mixture is successively washed with a solution of 0.1 N HCl, water, a 5% sodium bicarbonate solution and finally water. The organic phase is evaporated to dryness and the oil obtained is solubilized in 400 mL of a 4M solution of HCl in dioxane. After stirring for 4 hours at ambient temperature, the reaction mixture is evaporated to dryness and crystallized from ethanol. AlaVE hydrochloride (33.8 g of a white powder) thus prepared is analyzed by proton NMR in CDCl₃ and exhibits a spectrum in accordance with its chemical structure.

Stage 3: Grafting of AlaVE onto the Purified Polyacrylic Acid

3.74 g of AlaVE are solubilized in 50 mL of DMF and 0.97 mL of triethylamine. In parallel, 10 g of purified Degacryl (stage 1) are dissolved in 250 mL of N,N-dimethylformamide (DMF) and 0.34 g of 4-dimethylaminopyridine (DMAP). This solution is cooled down to 15° C., and the suspension of AlaVE/triethylamine then 1.93 g of N,N′-Diisopropylcarbodiimide (DIPC) are added successively. The reaction mixture is stirred overnight at 15° C. After the addition of a 35% HCl solution (1.16 mL) diluted in 3 mL of DMF, the reaction mixture is neutralized with 1 N soda in 900 mL of water. The solution obtained is diafiltered against 8 volumes of salt water (0.9% NaCl), then 4 volumes of water, and concentrated to a volume of approximately 400 mL. 100 mL of ethanol are added, the solution obtained is stirred overnight at ambient temperature, then diafiltered against 8 volumes of water and concentrated to a concentration of approximately 45 g/L.

The percentage of grafted AlaVE, determined by proton NMR in TFA-d, is 5.3%. The size of the particles, measured by light diffraction is 17 nm. The average molar mass is 37 kDa (PMMA equivalents) and the polydispersity index is 2.6.

### Example 2

### Synthesis of Polymer 2: Polyacrylic Acid Substituted with Approximately 8 Molar % of Alpha-Tocopherol Grafts Linked Via Alanine

1.20 g of AlaVE (stage 2 of Example 1) is solubilized in 10 mL of DMF and 0.31 mL of triethylamine. In parallel, 2 g of purified Degacryl (stage 1 of Example 1) are solubilized in 50 mL of DMF and 0.14 g of DMAP. This solution is cooled down to 15° C. and the suspension of AlaVE/triethylamine then 0.49 g of DIPC are added successively. The reaction mixture is stirred overnight at 15° C. After the addition of a 35% HCl solution (0.23 mL) diluted in 2 mL of DMF, the reaction mixture is stirred for 1 hour, then 40 mL of ethanol are added. This mixture is neutralized with 1 N soda in 110 mL of water then the suspension obtained is successively dialyzed against salt water (0.9% NaCl) then water and finally concentrated to 250 mL. 110 mL of ethanol are added, and the mixture is heated at 45° C. for 2 hours, then stirred overnight at ambient temperature. The solution obtained is diafiltered against 8 volumes of salt water (0.9%), then 4 volumes of water, and finally concentrated to a volume of approximately 20 mL.

The percentage of grafted AlaVE, determined by proton NMR in TFA-d, is 7.8%. The size of the particles, measured by light diffraction, is 12 nm. The average molar mass is 39 kDa (PMMA equivalents) and the polydispersity index is 2.9.

### Example 3

### Synthesis of Polymer 3: Polyacrylic Acid Substituted with about 10 Molar % of Alpha-Tocopherol Grafts Linked Via Alanine

Stage 1: Purification of Commercial Polyacrylic Acid (Acumer 1100)

100 g of Acumer 1100 (sold by Rohm and Haas) are diluted with 1000 g of milli-Q water. The solution is adjusted at pH=1.9 with a 1 N HCl solution, then diafiltered against 8 volumes of water. The solution obtained is then freeze-dried. The average molar mass Mn, measured by steric exclusion chromatography, is 14.5 kDa in PMMA (polymethyl methacrylate) equivalent and the polydispersity index is 1.27.

Stage 2: Grafting of AlaVE onto the Purified Polyacrylic Acid

7.48 g of AlaVE are solubilized in 190 mL of DMF and 1.95 mL of triethylamine. In parallel, 10 g of purified Acumer (stage 1) are dissolved in 250 mL of N,N-dimethylformamide (DMF) and 0.85 g of 4-dimethylaminopyridine (DMAP). This solution is cooled down to 15° C., and the suspension of AlaVE/triethylamine then 2.98 g of N,N′-Diisopropylcarbodiimide (DIPC) are added successively. The reaction mixture is stirred overnight at 15° C. After the addition of a 35% HCl solution (1.16 mL) diluted in 12 mL of DMF, the reaction mixture is neutralized with 1 N soda in 730 mL of water. The solution obtained is purified by diafiltration and concentrated to a volume of approximately 400 mL.

The percentage of grafted AlaVE, determined by proton NMR in TFA-d, is 9.9%. The size of the particles, measured by light diffraction is 10 nm. The average molar mass is 15.2 kDa (PMMA equivalents) and the polydispersity index is 1.29.

### Example 4

### Synthesis of Polymer 4: Polyacrylic Acid Substituted with about 20 Molar % of Alpha-Tocopherol Grafts Linked Via Alanine

14.96 g of AlaVE are solubilized in 380 mL of DMF and 3.87 mL of triethylamine. In parallel, 10 g of purified Acumer (stage 1) are dissolved in 250 mL of N,N-dimethylformamide (DMF) and 1.70 g of 4-dimethylaminopyridine (DMAP). This solution is cooled down to 15° C., and the suspension of AlaVE/triethylamine then 4.73 g of N,N′-Diisopropylcarbodiimide (DIPC) are added successively. The reaction mixture is stirred overnight at 15° C. After the addition of a 35% HCl solution (1.16 mL) diluted in 12 mL of DMF, the reaction mixture is neutralized with 1 N soda in 1,040 mL of water. The solution obtained is purified by diafiltration and concentrated to a volume of approximately 68 mL.

The percentage of grafted AlaVE, determined by proton NMR in TFA-d, is 18.5%. The size of the particles, measured by light diffraction is 13 nm. The average molar mass is 13.3 kDa (PMMA equivalents) and the polydispersity index is 1.28.

### Example 5

### Synthesis of a Polymer C1 not According to the Invention: the Same Polyacrylic Acid as Example 1, Substituted with Approximately 5 Molar % of Octadecylamine

3 g of purified Degacryl (stage 1 of Example 1) are solubilized in 75 mL of DMF and 0.10 g of DMAP, cooled down to 15° C. Then a suspension of 0.56 g of octadecylamine in 18.5 mL of DMF and 0.58 g of DIPC are added successively. The reaction mixture is stirred for 2 hours at 15° C. then overnight at 20° C. After the addition of 35% HCl (0.35 mL) diluted in 2 mL of DMF, the reaction mixture is neutralized with 1 N soda in a body of water of 250 mL. The solution obtained is diafiltered against 8 volumes of salt water (0.9%), then 4 volumes of water, and concentrated to a volume of approximately 100 mL. 20 mL of ethanol are added, and the solution obtained is stirred overnight at ambient temperature, then diafiltered against 8 volumes of water and concentrated to a concentration of approximately 25 g/L.

The percentage of grafted octadecylamine, determined by proton NMR in DMSO-d6, is 5.7%. The average molar mass is 38 kDa (PMMA equivalents) and the polydispersity index is 2.5.

### Example 6

### Measurement of the Viscosity (mPa/s) Under Shear of an Aqueous Solution with a Velocity Gradient of 10 s⁻¹

The viscosities of the aqueous solutions comprising respectively the polymer 1 of Example 1 and the polymer C1 of Example 5 described previously are measured under shear at a velocity gradient of 10 s⁻¹ (measurement thermostatically-controlled at 20° C. with a cone-plan geometry, 40 mm cone inclined by 1°, on a Gemini 150 device from Bohlin). These measurements are shown in Table 1 below.

TABLE 1

| Example | Concentration | Viscosity |
|---|---|---|
| Polymer 1 (invention) | 30 mg/g | 6 mPa/s |
| | 44 mg/g | 11 mPa/s |
| Polymer C1 (not according | 28 mg/g | 222 mPa/s |
| to the invention) | | |

The results show that the polymer 1 of the invention is far less viscous at the same grafting rate than the polymer C1. It remains easy to use (syringeability, homogeneous combination with an active ingredient) at much higher concentrations.

### Example 7

### Study of the Paclitaxel Combination

Increasing quantities of paclitaxel are added to vials containing 2 mL of polymer, and stirring is maintained at 25° C. for 12 hours. The transparency of the solution is then visually assessed. The compound is completely solubilized when the solution is transparent and reaches its solubility limit with a cloudy appearance. The solubility of the paclitaxel in the polymer solution (expressed in mg/g of polymer) is then expressed in the form of a low limit value. The first insolubility value is consequently the high limit.

The results are given in Table 2 below.

TABLE 2

| | | | |
|---|---|---|---|
| | | | Solubility |
| | | | of the |
| | Concentration | | paclitaxel: |
| | mg of polymer/g | Viscosity | mg/g |
| Example | of solution | mPa/s | polymer |

| | | | | |
|---|---|---|---|---|
| 1 | 44 | 11 | 61 | 68 |

The results show that the polymer 1 of the invention effectively makes it possible to solubilize a significant quantity of paclitaxel (per g of polymer). The solubility of the paclitaxel which is less than 0.25 μg/mL in water, can be increased to at least 2.7 mg/mL for a polymer solution at 44 mg/mL.

### Example 8

### Study of the Combination of Polymer 1 (Example 1) with Carvedilol

The polymer 1 of Example 1 (10 mg/mL) is dissolved with carvedilol (4 mg, base form) and the mixture is subjected to ultrasound for an hour then left under rotary stirring overnight. The quantity of carvedilol in solution after centrifugation is measured by UV spectroscopy. Solubilization of approximately 3 mg per 10 mg of polymer in solution is achieved, i.e. solubilization of 3 g/L. The base form carvedilol is soluble only at 50 mg/L in pure water.

### Example 9

### Study of the Combination of Polymer 2 (Example 2) with Ketoconazole

Polymer 2 of Example 2 (20 mg/g) is dissolved with ketoconazole (from 1 to 5 mg/g) and the mixtures are subjected to ultrasound for one hour then left under rotary stirring overnight. The transparency of the solution is then visually assessed. The compound is completely solubilized when it is transparent and reaches its solubility limit with a cloudy appearance. The low solubility limit is 3 mg/g i.e. approximately 3 g/L whereas the solubility of the ketoconazole in pure water is only 10 mg/L.

### Example 10

### Study of the Combination of Polymer 2 (Example 2) with Insulin

An aqueous solution containing 10 mg of polymer per millilitre at pH 7.4 and 400 IU of insulin (14.8 mg) is prepared. The solutions are left to incubate for 1 hour 30 minutes at 25° C. under stirring and the free insulin is separated from the insulin combined by ultrafiltration (threshold at 100 kDa, 15 minutes under 10,000 G at 18° C.). The free insulin recovered in the filtrate is then assayed by HPLC (High Performance Liquid Chromatography) and the quantity of combined insulin is deduced. The combined insulin is equal to 97%.

1. Polymer having a linear backbone of acrylic and/or methacrylic type to which alpha-tocopherol grafts are linked, characterized in that said alpha-tocopherol grafts are linked to said backbone via a spacer partly formed by at least one hydrolyzable function, and in that the distribution of said grafts on said backbone is random.

2. Polymer according to claim 1, characterized in that the molar grafting rate of alpha-tocopherol groups is less than or equal to 30 molar %.

3. Polymer according to any one of the previous claims, characterized in that it is capable of spontaneously forming nanoparticles when it is dispersed in an aqueous medium with a pH ranging from 5 to 8, in particular water.

4. Polymer according to the previous claim, characterized in that the size of the nanoparticles varies from 1 to 1,000 nm, in particular from 5 to 500 nm, in particular from 10 to 300 nm and more particularly from 10 to 200 nm or even 10 to 100 nm.

5. Polymer according to any one of the previous claims, characterized in that said spacer is an amino acid residue, preferably a natural amino acid residue.

6. Polymer according to any one of the previous claims, characterized in that it has the following formula (I), 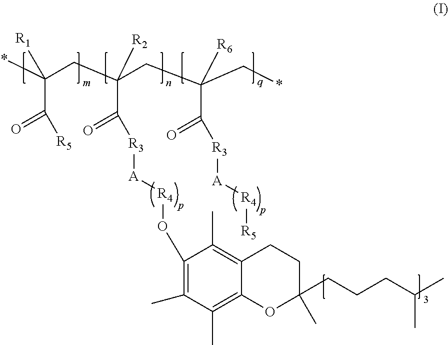
In which:
R₁, R₂ and R₆ represent independently an H or a methyl; —R₃-A-(R₄)ₚ— constitutes the spacer according to the invention;
R₃ represents —NH— or —O—;
A represents, a linear C₁ to C₂ alkyl or a linear or branched C₂ to C₆ alkyl or a methylene substituted by a benzyl group;
p is equal to 0 or 1, and preferably p is equal to 1;
R₄ represents C═O, O—C═O, or NH—C═O;
R₅ represents —OH or —OM, with M representing a cation, or R₅ represents a polyalkylene glycol substituent linked to the polymer via an ester function or an amide function;
m and n are positive integers;
q is equal to 0 or a positive integer, and preferably q is equal to 0;
(m+n+q) varies from 20 to 300,000;
the molar grafting rate of the alpha-tocopherol groups, n/(m+n+q) is less than or equal to 30 molar %,
the order of succession of two, or even three types of units forming the backbone of formula (I) being completely random.

7. Polymer according to any one of the previous claims, characterized in that
the spacer is an alanine residue.

8. Polymer according to any one of the previous claims, characterized in that it has a molar mass by weight ranging from 2,000 to 1,000,000, preferably from 5,000 to 50,000.

9. Polymer according to any one of the previous claims, characterized in that it also bears at least one graft of polyalkylene glycol type, in particular polyethylene glycol, and more particularly utilized with a molar percentage of grafting varying from 1 to 10%.

10. Method for the preparation of a polymer as defined according to any one of claims 1 to 9, characterized in that it comprises at least bringing, under conditions favourable to their interaction, at least one (meth)acrylic (co)polymer into contact with at least one alpha-tocopherol derivative functionalized with a spacer provided at its free end with a function capable of interacting with an acid function of said polymer, said spacer being such that, on completion of the reaction with said (co)polymer, it comprises at least one hydrolyzable function.

11. Method according to the previous claim, characterized in that the alpha-tocopherol derivative is functionalized by a spacer possessing at its free end a primary amine function, forming an amide bond after grafting to said (meth)acrylic type (co)polymer.

12. Composition characterized in that it comprises at least one polymer as defined in any one of claims 1 to 9.

13. Composition according to the previous claim, characterized in that it comprises at least one active ingredient, in particular an active ingredient of low or average aqueous solubility, said active ingredient being present there in a form non-covalently combined with nanoparticles formed by at least one polymer as defined according to any one of claims 1 to 9.

14. Composition according to claim 12 or 13, characterized in that it comprises at least one active ingredient of peptide or protein type.

15. Composition according to one of claims 12 to 14, in which said active ingredient is a molecule of therapeutic, cosmetic, prophylactic, or imaging interest.

16. Composition according to any one of claims 12 to 15, said composition being capable of ensuring a regulated release profile of said active ingredient as a function of time.

17. Composition according to any one of claims 12 to 16, in which said nanoparticles are agglomerated in the form of microparticles.

18. Composition according to any one of claims 12 to 17, in which said nanoparticles are utilized in the form of microparticles, said microparticles possessing a core containing said nanoparticles and at least one coating layer influencing a regulated release profile of said active ingredient as a function of the pH, said coating layer being formed by a material comprising at least one polymer A which is insoluble in water at a pH less than 5 and soluble in water at a pH greater than 7, combined with at least one hydrophobic compound B.

19. Composition according to the previous claim, in which polymer A is chosen from the methacrylic acid and methyl methacrylate copolymer(s), methacrylic acid and ethyl acrylate copolymer(s), cellulose acetate phthalate, cellulose acetate succinate, cellulose acetate trimellilate, hydroxypropylmethylcellulose phthalate, hydroxypropylmethylcellulose acetate succinate, carboxymethylethylcellulose, shellac gum, polyvinyl acetate phthalate, and mixtures thereof.

20. Composition according to any one of claims 18 and 19, in which the hydrophobic compound B is selected from the products crystallized in the solid state, and having a melting temperature T_{fb}≧40° C., preferably T_{fb}≧50° C., and still more preferably 40° C.≦T_{fb}≦90° C. and is more particularly chosen from the:
vegetable waxes;
hydrogenated vegetable oils alone or in a mixture with each other; preferably chosen froth the group comprising: hydrogenated cotton seed oil, hydrogenated soya oil, hydrogenated palm oil and mixtures thereof;
mono and/or di and/or tri esters of glycerol and of at least one fatty acid, preferably behenic acid;
and mixtures thereof.

21. Composition according to any one of claims 18 and 19, in which compound B is a polymer which is insoluble in water, and more particularly chosen from ethylcellulose, cellulose acetate butyrate, cellulose acetate, ammonio (meth)acrylate copolymers, poly(meth)acrylic acid esters, and mixtures thereof.

22. Composition according to any one of claims 12 and 21 formulated in the state of a powder, a solution, a suspension, or in the form of a tablet or a gelatin capsule.

23. Composition according to any one of claims 12 to 22, characterized in that it is intended for the preparation of medicaments.

24. Use of nanoparticles of at least one polymer as defined according to any one of claims 1 to 9, non-covalently combined with an active ingredient, in particular an active ingredient of low or average aqueous solubility, with a view to conveying, solubilizing and/or increasing the aqueous solubilization of said active ingredient.
